(19) United States
(12) Patent Application Publication
(10) Pub. No.: US 2026/0354751 A1
(43) Pub. Date: Nov. 12, 2026

(54) SLEEP RHYTHM DISORDER DETECTION METHOD AND APPARATUS BASED ON LONG-TERM SIGNALS AND MULTI-FEATURE EXTRACTION

(71) Applicant: SOUTHEAST UNIVERSITY, Nanjing (CN)

(72) Inventors: Chenxi YANG, Nanjing (CN); Yankun YAO, Nanjing (CN); Chang YAN, Nanjing (CN); Jianqing LI, Nanjing (CN); Chengyu LIU, Nanjing (CN)

(21) Appl. No.: 19/707,569

(22) Filed: Jun. 15, 2026

Related U.S. Application Data
(63) Continuation of application No. PCT/CN2025/142739, filed on Dec. 16, 2025.

(30) Foreign Application Priority Data
Dec. 16, 2024 (CN) 2024118496838

(51) Int. Cl. A61B 5/00

(52) U.S. Cl. CPC A61B 5/4806; A61B 5/6802; A61B 5/7225; A61B 5/7267

(57) ABSTRACT

A method and apparatus for detecting sleep rhythm disorder based on long-term signals and multi-feature extraction are disclosed. Physiological signals of a target user, including at least heart rate, are acquired, preprocessed, and subjected to peak extraction. Multi-domain heart rate variability features are extracted from the preprocessed physiological signals and combined with an individual's MEQ type to construct a multi-domain feature set. The multi-domain feature set is input into a trained sleep rhythm state evaluation model to output an individualized sleep rhythm disorder risk category or warning. Incorporating the MEQ type enables personalized rhythm disorder assessment while reducing workloads associated with frequent acquisition of physiological signals.

### TECHNICAL FIELD

The present disclosure relates to the technical field of medical signal processing, and in particular, to a method and an apparatus for detecting sleep rhythm disorder based on long-term signals and multi-feature extraction.

### BACKGROUND

Physiological activities of the human body follow certain periodic variation patterns, such as sleep, wakefulness, heart rate, emotion, and cognition, and exhibit regular variations during day-night cycles. Such a circadian rhythm is an adaptive response of the human body to the living environment, where sleep and wakefulness are the most fundamental rhythmic characteristics among fluctuations of all physiological functions. However, with social development, an accelerated pace of life, and increasing competitive pressure, prolonged exposure to stress conditions may lead to sleep rhythm disorders. Such disorders may manifest as a lack of coordination between sleep-wake timing and natural circadian rhythms, or as non-physiological sleep phenomena such as difficulty in falling asleep, insufficient sleep, or sleep phase disorders. Sleep rhythm disorders may severely affect physical and mental health, work capability, and quality of life, and are closely associated with the onset of many acute and chronic diseases. Long-term sleep rhythm disorders may result in emotional dysregulation, physical and mental fatigue, reduced immunity, and impaired attention and behavioral performance. In high-risk industries, such conditions may cause cognitive impairments such as reduced judgment ability and operational errors among workers, thereby leading to accidental casualties.

Currently, for risk prediction of sleep rhythm disorders, most existing medical devices and wearable intelligent devices merely provide quantitative display of sleep rhythms, whereas risk prediction of rhythm disorders generally requires manual estimation and analysis, and existing devices lack efficient data processing and analysis capabilities, thereby failing to provide real-time and more accurate risk warning for rhythm disorders. In addition, existing methods fail to combine an individual's sleep rhythm type, such as an MEQ type, with long-term heart rate variability features for personalized rhythm disorder assessment. Furthermore, since massive physiological signal data generated under continuous acquisition conditions impose substantial challenges on signal processing, there is an urgent need for a rhythm disorder risk prediction method applicable to long-term acquisition conditions and capable of taking individual sleep chronotype differences into account.

### SUMMARY

An objective of the present disclosure is to provide a method and an apparatus for detecting sleep rhythm disorder based on long-term signals and multi-feature extraction.

The objective of the present disclosure can be achieved by the following technical solutions:

In the present disclosure, the extracted multi-domain heart rate variability features are combined with an individual's MEQ type, which represents the individual's sleep chronotype, to construct a multi-domain feature set. By incorporating the MEQ type as an individual characteristic into the feature set used by the sleep rhythm state evaluation model, the method supports personalized rhythm disorder risk assessment for different sleep rhythm types.

A method for detecting sleep rhythm disorder based on long-term signals and multi-feature extraction is provided, and the method includes:
step S1: acquiring physiological signals of a target user, where the physiological signals at least include heart rate;
step S2: preprocessing the physiological signals and performing peak extraction thereon;
step S3: extracting multi-domain heart rate variability features from the preprocessed physiological signals by using a multi-domain feature extraction algorithm based on the processed signals;
step S4: constructing a multi-domain feature set by combining the extracted multi-domain heart rate variability features with an individual's MEQ type; and
step S5: inputting the multi-domain feature set into a trained sleep rhythm state evaluation model, and outputting an individualized sleep rhythm disorder risk category or warning.

The physiological signals are acquired by a wearable electronic device.

The preprocessing process in the step S2 includes removing high-frequency noise and low-frequency noise in the acquired physiological signals by using a band-pass filter.

The multi-domain heart rate variability features include HRV time-domain features, HRV frequency-domain features, and HRV nonlinear features, and the HRV nonlinear features include heart rate asymmetry features, Poincaré plot features, and a plurality of entropy complexities.

The Poincaré plot features include a Poincaré plot semi-major axis SD1, a semi-minor axis SD2, and a ratio SD1/SD2 of the semi-major axis to the semi-minor axis;
the plurality of entropy complexities include approximate entropy ApEn, sample entropy SampE, fuzzy entropy FuzzyEn, fuzzy measure entropy FuzzyMEn, and distribution entropy DistEn; and
the heart rate asymmetry features include: a Porta index, a Guzik index, a slope index, and an area index.

The step S4 includes:
combining the extracted multi-domain heart rate variability features with an individual's MEQ type, as an individual sleep chronotype characteristic, to construct a multi-domain feature set D, and preprocessing the feature set, where preprocessing of the feature set includes processing missing values and normalizing features.

The individual's MEQ type is a sleep rhythm type, including: a morning type, an evening type, and an intermediate type.

A method for constructing the trained sleep rhythm state evaluation model includes: selecting the Gini index as a splitting criterion for constructing a decision tree, where the Gini index Gini(D,a) of each feature of a dataset is calculated as follows:

Gini(D,a)=sum_v(|Dv|/|D|)Gini(Dv)

Gini(Dv)=1−sum_{k=1}\hat{ }{n}p_k\hat{ }2

where p_k represents a probability of different rhythm disorder risk categories in the dataset, n represents the number of rhythm disorder risk categories, Gini(Dv) represents the Gini coefficient under each feature, a represents a feature category, and Dv represents a feature set split based on feature a;
starting from a root node, calculating Gini indices of all features, and selecting a feature having a minimum Gini index and a corresponding value thereof as an optimal feature and an optimal threshold; and dividing the dataset into two subsets based on the selected optimal feature and optimal threshold, and then recursively repeating the above steps for each subset, that is, selecting another optimal feature and a threshold thereof for further splitting until a stopping condition is satisfied, where the stopping condition includes: all samples belonging to a same category, no available feature for further splitting, reaching a preset maximum tree depth, or the number of samples in a subset being less than a preset threshold.

The method for constructing the trained sleep rhythm state evaluation model further includes:
randomly dividing the feature set according to a preset ratio to obtain a training set and a test set, inputting the test set into a trained sleep rhythm state evaluation model for result prediction, where the sleep rhythm state evaluation model is constructed by integrating decision trees, and model accuracy evaluation is performed by using accuracy, a confusion matrix, and ten-fold cross-validation.

An apparatus for detecting sleep rhythm disorder based on long-term signals and multi-feature extraction is provided, and the apparatus includes a physiological-signal acquisition unit, an output unit, a memory, and a processor, where the physiological-signal acquisition unit is configured to acquire physiological signals of a target user, the physiological signals at least including heart rate, the processor, when executing a program stored in the memory, implements the method described above, and the output unit is configured to output the individualized sleep rhythm disorder risk category or warning.

Compared with the prior art, the present disclosure has the following beneficial effects:
1. The sleep rhythm of a target user is monitored and analyzed based on long-term signals, which facilitates real-time capture of potential rhythm disorders under long-term sleep conditions and reduces workloads associated with frequent acquisition of physiological signals.
2. The method analyzes rhythm disorder risks from multiple perspectives through multi-feature extraction of long-term signals, in combination with multi-domain heart rate variability features and an individual's MEQ type. In particular, the MEQ type is used as an individual sleep chronotype characteristic and is combined with the HRV feature set so that the sleep rhythm state evaluation model can perform chronotype-aware personalized assessment. Heart rate asymmetry features, Poincaré plot features, and a plurality of entropy complexities are introduced into the multi-domain heart rate variability features, thereby improving the accuracy of rhythm disorder risk prediction.
3. When applied in daily life and operational activities, the method can assist in providing individualized warning information for potential rhythm disorder risks. In one aspect, potential rhythm disorder risks of individuals can be identified to support risk assessment. In another aspect, the warning information may support personnel management and operational safety during production operations by providing information associated with fatigue and inattention resulting from rhythm disorders.

### BRIEF DESCRIPTION OF THE DRAWINGS

FIG. 1 is a schematic flowchart of main steps of the method according to the present disclosure; and

FIG. 2 is a scatter plot of RP intervals.

### DETAILED DESCRIPTION

The present disclosure will be described in detail below with reference to the drawings and specific embodiments. The embodiments are implemented on the premise of the technical solutions of the present disclosure, and a detailed implementation and a specific operation process are provided, but the scope of the present disclosure is not limited to the following embodiments.

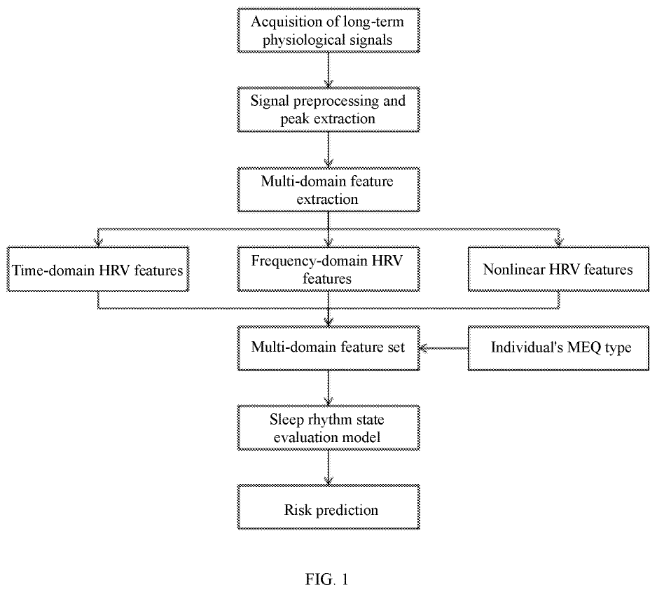

A method for detecting sleep rhythm disorder based on long-term signals and multi-feature extraction is provided. As shown in FIG. 1, the method includes the following steps.

In step S1, physiological signals of a target user are acquired, where the physiological signals at least include heart rate.

The physiological signals are acquired by a wearable electronic device. Generally, the wearable electronic device may be a non-invasive electronic device such as an electrocardiogram patch or a pulse sensor, which is capable of acquiring physiological data of the target user in real time. Specifically, the physiological signals mainly include heart rate, and may further be combined with pulse data. Certainly, in some embodiments, the pulse sensor may be a blood oxygen pulse sensor.

In step S2, the physiological signals are preprocessed and peak extraction is performed on the physiological signals.

The preprocessing process includes removing high-frequency noise and low-frequency noise from the acquired physiological signals by using a band-pass filter, and the specific process includes: setting a band-pass filter having a frequency range of 0.5 Hz to 4 Hz to remove low-frequency noise and high-frequency noise from the acquired physiological signals.

In addition, in the embodiments, peak feature extraction is performed on the processed data by using a Pan-Tompkins algorithm.

In step S3, multi-domain heart rate variability features are extracted from the preprocessed physiological signals by using a multi-domain feature extraction algorithm based on the processed signals.

The multi-domain heart rate variability features include HRV time-domain features, HRV frequency-domain features, and HRV nonlinear features, and the HRV nonlinear features include heart rate asymmetry features, Poincaré plot features, and a plurality of entropy complexities.

The HRV time-domain features include a standard deviation of normal sinus RR intervals (SDNN), a root mean square of differences between adjacent RR intervals (RMSSD), a percentage of adjacent RR interval differences greater than 50 ms among all RR intervals (pNN_50), and the like. The specific calculation methods are as follows:

SDNN=sqrt(sum_{i=1}\hat{ }{N}(RR_i−meanRR)\hat{ }2/(N−1))

RMSSD=sqrt(sum_{i=1}\hat{ }{N}(RR_{i+1}−RR_i)\hat{ }2/N)

pNN_50=(NN_50/N)×100

where N represents the total number of normal heartbeats during the entire recording, meanRR represents an average value of RR intervals of the N heartbeats; RRi and RRi+1 represent durations of two adjacent sinus cardiac cycles; NN_50 represents the number of adjacent RR interval differences greater than 50 ms during the entire recording, and N represents the total number of RR intervals.

The HRV frequency-domain features include total power (TP), low-frequency power (LF), high-frequency power (HF), a low-frequency/high-frequency ratio (LF/HF), and the like. The specific calculation methods are as follows:

Power spectral analysis of RR interval series is performed using an AR model. An area within a range of 0-0.4 Hz obtained therefrom is defined as the total power TP. An area within a range of 0.04-0.15 Hz obtained therefrom is defined as the low-frequency power LF, and an area within a range of 0.15-0.4 Hz obtained therefrom is defined as the high-frequency power HF.

The Poincaré plot features include a Poincaré plot semi-major axis SD1, a semi-minor axis SD2, and a ratio SD1/SD2 of the semi-major axis to the semi-minor axis. The specific calculation method is as follows:

SD1=sqrt(D((sqrt(2)/2)(Y−X)))=STD(Y−X)/sqrt(2)

SD2=sqrt(D((sqrt(2)/2)(X+Y)))=STD(X+Y)/sqrt(2)

where X={xᵢ|i=1 . . . N}, Y={yᵢ|i=1 . . . N}, and (xi, yi) represent coordinates of points in the Poincaré scatter plot.

The plurality of entropy complexities include approximate entropy ApEn, sample entropy SampE, fuzzy entropy FuzzyEn, fuzzy measure entropy FuzzyMEn, and distribution entropy DistEn.

The heart rate asymmetry features include: a Porta index, a Guzik index, a slope index, and an area index.

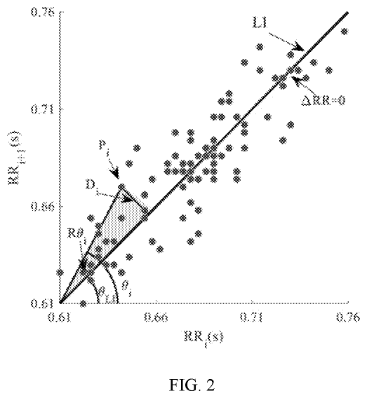

For RR intervals (RR₁, RR₂, . . . , RRᵢ, RRᵢ₊₁, . . . , RRₙ₋₁, RRₙ), as shown in FIG. 2, an algorithm for the heart rate asymmetry indices implemented by the following formulas is described, where LI represents an identity line y=x, Pᵢ represents the i-th point and may be represented as (RRᵢ, RRᵢ₊₁), ΔRR=RRᵢ₊₁−RRᵢ, Dᵢ represents the distance from Pᵢ to LI, an included angle between a line connecting the coordinate origin and the horizontal coordinate is represented as θᵢ, and the phase angle between the horizontal coordinate and LI is represented as θ_{LI}.

For the Porta index, abbreviated as PI, PI measures a proportion of points below LI.

The formula for calculating PI is as follows:

PI=(b/m)×100

where b and m respectively represent the number of points below L1 and the number of points not located on L1.

For the Guzik index, abbreviated as GI, GI represents a ratio based on cumulative distances of points and can be calculated as follows:

GI=(sum_{i=1}\hat{ }{l}D_i/sum_{i=1}\hat{ }{m}D_i)×100

D_i=|RR_{i+1}−RR_i|/sqrt(2)

For the slope index, abbreviated as SI, SI evaluates the percentage of cumulative phase angles above LI and can be calculated as follows:

SI=(sum_{i=1}\hat{ }{l}|Rθ_i|/sum_{i=1}\hat{ }{m}|Rθ_i|)×100

where Rθ_i=θ_LI−θ_i.

For the area index, abbreviated as AI, the area of the i-th sector is represented as:

S_i=(½)×Rθ_i×r\hat{ }2

where r represents the distance from Pi to the coordinate origin.

Then, AI is defined as the ratio of Si above LI, and the formula of AI is as follows:

AI=(sum_{i=1}\hat{ }{l}|S_i|/sum_{i=1}\hat{ }{m}|S_i|)×100

In step S4, multi-domain feature set construction and rhythm disorder risk classification are performed on the extracted multi-domain heart rate variability features. This step includes:
combining the extracted multi-domain heart rate variability features with an individual's MEQ type, as an individual sleep chronotype characteristic, to construct a multi-domain feature set D, and preprocessing the feature set, where preprocessing of the feature set includes processing missing values and normalizing features. In the embodiments, z-score standardization is employed for data normalization.

The individual's MEQ type is a sleep rhythm type, including: a morning type, an evening type, and an intermediate type. By combining the individual's MEQ type with the multi-domain heart rate variability features, the multi-domain feature set D reflects both long-term physiological rhythm information and individual sleep chronotype information.

In the embodiments, the individual's rhythm disorder risk category is classified based on the individual's PSQI score. A PSQI score greater than 8 indicates a high risk of rhythm disorder, and a PSQI score lower than 8 indicates a low risk of rhythm disorder.

In step S5, the multi-domain feature set is input into a trained sleep rhythm state evaluation model, and an individualized sleep rhythm disorder risk category or warning is output.

The trained sleep rhythm state evaluation model is constructed by using a random forest algorithm. The method for constructing the trained sleep rhythm state evaluation model includes:
selecting the Gini index as a splitting criterion for constructing a decision tree, where the Gini index Gini(D,a) of each feature of a dataset is calculated as follows:

Gini(D,a)=sum_v(|Dv|/|D|)Gini(Dv)

Gini(Dv)=1−sum_{k=1}\hat{ }{n}p_k\hat{ }2

where p_k represents a probability of different rhythm disorder risk categories in the dataset, n represents the number of rhythm disorder risk categories, Gini(Dv) represents the Gini coefficient under each feature, a represents a feature category, and Dv represents a feature set split based on feature a;
starting from a root node, calculating Gini indices of all features, and selecting a feature having a minimum Gini index and a corresponding value thereof as an optimal feature and an optimal threshold; and dividing the dataset into two subsets based on the selected optimal feature and optimal threshold, and then recursively repeating the above steps for each subset, that is, selecting another optimal feature and a threshold thereof for further splitting until a stopping condition is satisfied, where the stopping condition includes: all samples belonging to a same category, no available feature for further splitting, reaching a preset maximum tree depth, or the number of samples in a subset being less than a preset threshold.

The method for constructing the trained sleep rhythm state evaluation model further includes:
randomly dividing the feature set according to a preset ratio to obtain a training set and a test set, inputting the test set into a trained sleep rhythm state evaluation model for result prediction, where the sleep rhythm state evaluation model is constructed by integrating decision trees, and model accuracy evaluation is performed by using accuracy, a confusion matrix, and ten-fold cross-validation.

In the embodiments, the random forest model repeats the above decision tree construction process 100 times to generate 100 decision trees. The 100 generated decision trees constitute a random forest. The mode of prediction results of the 100 decision trees is used as the final output result of the random forest prediction model.

In the embodiments, the multi-domain feature set is divided into a training set and a test set according to a ratio of 7:3, the test set is input into the random forest model for result prediction, and model accuracy evaluation is performed by using accuracy, a confusion matrix, and ten-fold cross-validation.

The functions described above may be stored in a non-transitory computer-readable storage medium if they are implemented in software functional units and sold or used as independent products. Based on such understandings, the technical solutions of the present disclosure substantially or a part thereof that contributes to the prior art, or a part of the technical solutions, may be embodied in a software product, which is stored in a non-transitory storage medium and includes several instructions for causing a computer device (which may be a personal computer, a server, a network device, or the like) to perform all or part of the steps of the methods as defined in the embodiments of the present disclosure. The aforementioned storage medium includes: a U-disk, a mobile hard disk, a read-only memory (ROM), a random access memory (RAM), a magnetic disk, or an optical disk, and various media capable of storing program codes.

1. A method for detecting sleep rhythm disorder based on long-term signals and multi-feature extraction, the method comprising:
acquiring physiological signals of a target user, wherein the physiological signals at least comprise heart rate;
preprocessing the physiological signals and performing peak extraction thereon;
extracting multi-domain heart rate variability features from the preprocessed physiological signals by using a multi-domain feature extraction algorithm based on the processed signals;
constructing a multi-domain feature set by combining the extracted multi-domain heart rate variability features with an individual's MEQ type;
inputting the multi-domain feature set into a trained sleep rhythm state evaluation model, and outputting an individualized sleep rhythm disorder risk category or warning,
wherein the multi-domain heart rate variability features comprise HRV time-domain features, HRV frequency-domain features, and HRV nonlinear features, and the HRV nonlinear features comprise heart rate asymmetry features, Poincaré plot features, and a plurality of entropy complexities;
wherein the Poincaré plot features comprise a Poincaré plot semi-major axis SD1, a semi-minor axis SD2, and a ratio SD1/SD2 of the semi-major axis to the semi-minor axis;
wherein the plurality of entropy complexities comprise approximate entropy ApEn, sample entropy SampE, fuzzy entropy FuzzyEn, fuzzy measure entropy FuzzyMEn, and distribution entropy DistEn;
wherein the heart rate asymmetry features comprise a Porta index, a Guzik index, a slope index, and an area index;
wherein constructing the multi-domain feature set comprises:
combining the extracted multi-domain heart rate variability features with the individual's MEQ type to construct a multi-domain feature set D, and preprocessing the feature set, wherein preprocessing of the feature set comprises processing missing values and normalizing features;
wherein the individual's MEQ type is a sleep rhythm type, comprising: a morning type, an evening type, and an intermediate type; and
wherein the trained sleep rhythm state evaluation model is constructed by a random forest algorithm, and constructing the trained sleep rhythm state evaluation model comprises:
selecting the Gini index as a splitting criterion for constructing a decision tree,
wherein the Gini index Gini(D, a) of each feature of a dataset is calculated as follows:
Gini(D,a)=sum_v(|Dv|/|D|)Gini(Dv)
Gini(Dv)=1−sum_{k=1}\hat{ }{n}p_k\hat{ }2
wherein p_k represents a probability of different rhythm disorder risk categories in the dataset, n represents the number of rhythm disorder risk categories, Gini(Dv) represents the Gini coefficient under each feature, a represents a feature category, and Dv represents a feature set split based on feature a;
starting from a root node, calculating Gini indices of all features, and selecting a feature having a minimum Gini index and a corresponding value thereof as an optimal feature and an optimal threshold; and
dividing the dataset into two subsets based on the selected optimal feature and optimal threshold, and recursively repeating the above steps for each subset by selecting another optimal feature and a threshold thereof for further splitting until a stopping condition is satisfied, wherein the stopping condition comprises: all samples belonging to a same category, no available feature for further splitting, reaching a preset maximum tree depth, or the number of samples in a subset being less than a preset threshold.

2. The method for detecting sleep rhythm disorder based on long-term signals and multi-feature extraction according to claim 1, wherein the physiological signals are acquired by a wearable electronic device.

3. The method for detecting sleep rhythm disorder based on long-term signals and multi-feature extraction according to claim 1, wherein the preprocessing process comprises removing high-frequency noise and low-frequency noise in the acquired physiological signals by using a band-pass filter.

4. The method for detecting sleep rhythm disorder based on long-term signals and multi-feature extraction according to claim 1, wherein constructing the trained sleep rhythm state evaluation model further comprises:
randomly dividing the feature set according to a preset ratio to obtain a training set and a test set, inputting the test set into the trained sleep rhythm state evaluation model for result prediction, wherein the trained sleep rhythm state evaluation model is constructed by integrating decision trees, and model accuracy evaluation is performed by using accuracy, a confusion matrix, and ten-fold cross-validation.

5. An apparatus for detecting sleep rhythm disorder based on long-term signals and multi-feature extraction, the apparatus comprising a physiological-signal acquisition unit, an output unit, a memory, and a processor, wherein the physiological-signal acquisition unit is configured to acquire physiological signals of a target user, the physiological signals at least comprising heart rate, wherein the processor, when executing a program stored in the memory, implements the method according to claim 1, and wherein the output unit is configured to output the individualized sleep rhythm disorder risk category or warning.
